(19) United States
(12) Patent Application Publication
(10) Pub. No.: US 2012/0029300 A1
(43) Pub. Date: Feb. 2, 2012

(54) SYSTEM AND METHOD FOR REDUCING FALSE ALARMS AND FALSE NEGATIVES BASED ON MOTION AND POSITION SENSING

(75) Inventor: Pierre Paquet, Quebec (CA)

(73) Assignee: CareFusion 303, Inc., San Diego, CA (US)

(21) Appl. No.: 12/844,765

(22) Filed: Jul. 27, 2010

(51) Int. Cl. A61B 5/00

(52) U.S. Cl. 600/300

(57) ABSTRACT

Systems and methods of reducing false alarms associated with vital-sign monitoring are disclosed. One or more vital-sign readings of a patient are received. One or more acceleration readings from an accelerometer attached to the patient are received. At least one of motion and position of the patient are determined based at least in part on the one or more acceleration readings. An alarm condition determination procedure is modified if at least one of the motion and the position satisfies a predefined condition.

### CROSS-REFERENCES TO RELATED APPLICATIONS

The following applications disclose certain common subject matter with the present application: A Vital-Signs Monitor with Encapsulation Arrangement, docket number 080624-0612; A Vital-Signs Monitor with Spaced Electrodes, docket number 080624-0623; A Vital-Signs Patch Having a Strain Relief, docket number 080624-0624; A Temperature Probe Suitable for Axillary Reading, docket number 080624-0625; A System and Method for Detecting Loss of Thermal Contact Between a Patient and a Temperature Probe, docket number 080624-0626; A System and Method for Storing and Forwarding Data from a Vital-Signs Monitor, docket number 080624-0627; A System and Method for Conserving Battery Power in a Patient Monitoring System, docket number 080624-0629; A System and Method for Saving Battery Power in a Patient Monitoring System, docket number 080624-0630; A System And Method for Tracking Vital-Signs Monitor Patches, Docket Number 080624-0631; A System And Method for Reducing False Alarms Associated with Vital-Signs Monitoring, Docket Number 080624-0632; A System And Method for Location Tracking of Patients in a Vital-Signs Monitoring System, Docket Number 080624-0633; all of the listed applications filed on ______.

### FIELD

The present disclosure generally relates to systems and methods of physiological monitoring, and, in particular, relates to systems and methods for reducing false alarms based on motion and position sensing.

### DESCRIPTION OF THE RELATED ART

Some of the most basic indicators of a person's health are those physiological measurements that reflect basic body functions and are commonly referred to as a person's “vital signs.” The four measurements commonly considered to be vital signs are body temperature, pulse rate, blood pressure, and respiratory rate. Most or all of these measurements are performed routinely upon a patient when they arrive at a healthcare facility, whether it is a routine visit to their doctor or arrival at an Emergency Room (ER).

Vital signs are frequently taken by a nurse using basic tools including a thermometer to measure body temperature, a sphygmomanometer to measure blood pressure, and a watch to count the number of breaths or the number of heart beats in a defined period of time, typically 10 seconds, which is then converted to a “per minute” rate. If a patient's pulse is weak, it may not be possible to detect a pulse by hand and the nurse may use a stethoscope to amplify the sound of the patient's heart beat so that she can count the beats.

When a patient is admitted to a hospital, it is common for vital signs to be measured and recorded at regular intervals during the patient's stay to monitor their condition. A typical interval is 4 hours, which leads to the undesirable requirement for a nurse to awaken a patient in the middle of the night to take vital sign measurements.

When a patient is admitted to an ER, it is common for a nurse to do a “triage” assessment of the patient's condition that will determine how quickly the patient receives treatment. During busy times in an ER, a patient who does not appear to have a life-threatening injury may wait for hours until more-serious cases have been treated. While the patient may be reassessed at intervals while awaiting treatment, the patient may not be under observation between these reassessments.

Measuring certain vital signs is normally intrusive at best and difficult to do on a continuous basis. Measurement of body temperature, for example, is commonly done by placing an oral thermometer under the tongue or placing an infrared thermometer in the ear canal such that the tympanic membrane, which shared blood circulation with the brain, is in the sensor's field of view. Other countries report temperatures made by placing a thermometer under the arm, referred to as an “axillary” measurement as axilla is the Latin word for armpit. Skin temperature can be measured using a stick-on strip that may contain panels that change color to indicate the temperature of the skin below the strip.

Measurement of respiration is easy for a nurse to do, but relatively complicated for equipment to achieve. A method of automatically measuring respiration is to encircle the upper torso with a flexible band that can detect the physical expansion of the rib cage when a patient inhales. An alternate technique is to measure a high-frequency electrical impedance between two electrodes placed on the torso and detect the change in impedance created when the lungs fill with air. The electrodes are typically placed on opposite sides of one or both lungs, resulting in placement on the front and back or on the left and right sides of the torso, commonly done with adhesive electrodes connected by wires or by using a torso band with multiple electrodes in the strap.

Measurement of pulse is also relatively easy for a nurse to do and intrusive for equipment to achieve. A common automatic method of measuring a pulse is to use an electrocardiograph (ECG or EKG) to detect the electrical activity of the heart. An EKG machine may use up to 10 electrodes placed at defined points on the body to detect various signals associated with the heart function. Another common piece of equipment is simply called a “heart rate monitor.” Widely sold for use in exercise and training, heart rate monitors commonly consist of a torso band, in which are embedded two electrodes held against the skin and a small electronics package. Such heart rate monitors can communicate wirelessly to other equipment such as a small device that is worn like a wrist watch and that can transfer data wirelessly to a PC.

Nurses are expected to provide complete care to an assigned number of patients. The workload of a typical nurse is increasing, driven by a combination of a continuing shortage of nurses, an increase in the number of formal procedures that must be followed, and an expectation of increased documentation. Replacing the manual measurement and logging of vital signs with a system that measures and records vital signs would enable a nurse to spend more time on other activities and avoid the potential for error that is inherent in any manual procedure.

### SUMMARY

For some or all of the reasons listed above, there is a need for a hospital or other caregiver facility to be able to continuously monitor its patients in different settings within the hospital. In addition, it is desirable for this monitoring to be done with limited interference with a patient's mobility or interfering with their other activities.

Embodiments of the patient monitoring system disclosed herein measure certain vital-sign readings of a patient, which can include respiratory rate, pulse rate, and body temperature, on a regular basis and compare these measurements to preset limits.

In addition, certain embodiments of the patient monitoring system disclosed herein can send alarm notifications to a hospital system if the vital-sign readings satisfy an alarm condition. It is desirable to reduce or eliminate false positives in the alarm notifications so as to avoid unnecessary trips or other actions of a healthcare provider that such false positives can cause.

In certain aspects of the present disclosure, a method of reducing false alarms associated with vital-sign monitoring is disclosed. The method comprises receiving one or more vital-sign readings of a patient. The method can further comprise receiving one or more acceleration readings from an accelerometer attached to the patient. The method can further comprise determining at least one of motion and position of the patient based at least in part on the one or more acceleration readings. The method can further comprise modifying an alarm condition determination procedure if at least one of the motion and the position satisfies a predefined condition.

In certain aspects of the present disclosure, a vital-sign monitoring system is provided that comprises a vital-sign monitor configured to monitor one or more vital signs of a patient. The system can further comprise a surveillance server configured to gather data relating to the one or more vital signs of the patient from the vital-sign monitor. The system can further comprise an accelerometer attached to the patient. The system can further comprise a processor in data communication with the vital-sign monitor and the accelerometer. The processor can be configured to receive one or more vital-sign readings of the patient, receive one or more acceleration readings from the accelerometer, determine at least one of motion and position of the patient based at least in part on the one or more acceleration readings, and modify an alarm condition determination procedure if at least one of the motion and the position satisfies a predefined condition.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1 is a diagram illustrating an exemplary embodiment of a patient monitoring system according to certain aspects of the present disclosure.

FIG. 2A is a perspective view of the vitals sign monitor patch shown in FIG. 1 according to certain aspects of the present disclosure.

FIG. 2B is a cross-sectional view of the vital signs patch shown in FIGS. 1 and 2A according to certain aspects of the present disclosure.

FIG. 2C is a functional block diagram illustrating exemplary electronic and sensor components of the monitor patch of FIG. 1 according to certain aspects of the present disclosure.

FIG. 3A is a functional schematic diagram of an embodiment of the bridge according to certain aspects of the present disclosure.

FIG. 3B is a functional schematic diagram of an embodiment of the surveillance server according to certain aspects of the present disclosure.

FIG. 4A is a diagram depicting a patient wearing a temperature monitoring system comprising a monitor patch and a temperature probe and configured to measure body temperature of the patient according to certain aspects of the present disclosure.

FIG. 4B is a diagram providing an enlarged view of the temperature monitoring system depicted in FIG. 4A according to certain aspects of the present disclosure.

FIG. 5 depicts a graph representing a series of data samples corresponding to a vital sign of a patient generated by a monitor patch according to certain aspects of the present disclosure.

FIG. 6 is a diagram depicting an exemplary vital-sign monitoring system that is configured to determine motion and/or position of a patient and thereby prevent or reduce false alarms associated with the patient motion/position according to certain aspects of the present disclosure.

FIG. 7 is a flowchart illustrating an exemplary process for determining motion or position of a patient and thereby preventing or reducing false alarms associated with the patient motion/position according to certain aspects of the present disclosure.

### DETAILED DESCRIPTION

Continuous monitoring of patients in a hospital is desirable at least to ensure that patients do not suffer an un-noticed sudden deterioration in their condition or a secondary injury during their stay in the hospital. It is impractical to provide continuous monitoring by a clinician and cumbersome to connect sensors to a patient, which are then connected to a fixed monitoring instrument by wires. Furthermore, systems that sound an alarm when the measured value exceeds a threshold value may sound alarms so often and in situations that are not truly serious that such alarms are ignored by clinicians.

Measuring vital signs is difficult to do on a continuous basis. Accurate measurement of cardiac pulse, for example, can be done using an electrocardiograph (ECG or EKG) to detect the electrical activity of the heart. An EKG machine may use up to 10 electrodes placed at various points on the body to detect various signals associated with the heart function. Another common piece of equipment is termed a “heart rate monitor.” Widely sold for use in exercise and physical training, heart rate monitors may consist of a torso band in which are embedded two electrodes held against the skin and a small electronics package. Such heart rate monitors can communicate wirelessly to other equipment such as a small device that is worn like a wrist watch that can transfer data wirelessly to a personal computer (PC).

Certain exemplary embodiments of the present disclosure include a system that comprises a vital-signs monitor patch that is attached to the patient, and a bridge that communicates with monitor patches and links them to a central server that processes the data, where the server also sends data and alarms to the hospital system according to algorithms and protocols defined by the hospital.

The construction of the vital-signs monitor patch is described according to certain aspects of the present disclosure. As the patch may be worn continuously for a period of time that may be several days, as is described in the following disclosure, it is desirable to encapsulate the components of the patch such that the patient can bathe or shower and engage in their normal activities without degradation of the patch function. An exemplary configuration of the construction of the patch to provide a hermetically sealed enclosure about the electronics is disclosed.

In the following detailed description, numerous specific details are set forth to provide a full understanding of the present disclosure. It will be apparent, however, to one ordinarily skilled in the art that embodiments of the present disclosure may be practiced without some of the specific details. In other instances, well-known structures and techniques have not been shown in detail so as not to obscure the disclosure.

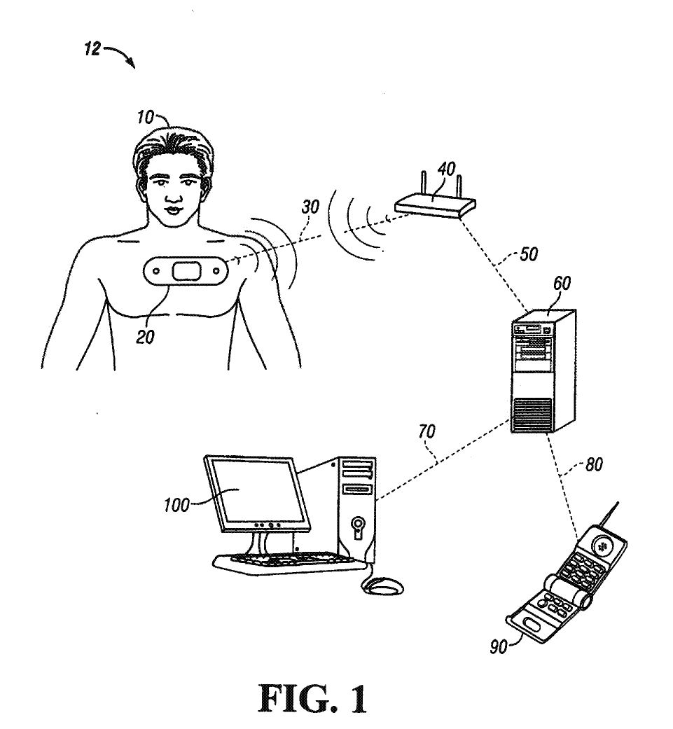

FIG. 1 discloses a vital sign monitoring system according to certain embodiments of the present disclosure. The vital sign monitoring system 12 includes vital-signs monitor patch 20, bridge 40, and surveillance server 60 that can send messages or interact with peripheral devices exemplified by mobile device 90 and workstation 100.

Monitor patch 20 resembles a large adhesive bandage and is applied to a patient 10 when in use. It is preferable to apply the monitor patch 20 to the upper chest of the patient 10 although other locations may be appropriate in some circumstances. Monitor patch 20 incorporates one or more electrodes (not shown) that are in contact with the skin of patient 10 to measure vital signs such as cardiac pulse rate and respiration rate. Monitor patch 20 also may include other sensors such as an accelerometer or a temperature sensor to measure other characteristics associated with the patient. Monitor patch 20 also includes a wireless transmitter that can both transmit and receive signals. This transmitter is preferably a short-range, low-power radio frequency (RF) device operating in one of the industrial, scientific and medical (ISM) radio bands. One ISM band in the United States (US) is, for example, centered at 915 MHz. An example of an equivalent band in the European Union (EU) is centered at 868 MHz. Other frequencies of operation may be possible dependent upon local regulations and interference from other wireless devices.

Surveillance server 60 may be a standard computer server connected to the hospital communication network and preferably located in the hospital data center or computer room, although other locations may be employed. The server 60 stores and processes signals related to the operation of the patient monitoring system 12 disclosed herein including the association of individual monitor patches 20 with patients 10 and measurement signals received from multiple monitor patches 20. Hence, although only a single patient 10 and monitor patch 20 are depicted in FIG. 1, the server 60 is able to monitor the monitor patches 20 for multiple patients 10.

Bridge 40 is a device that connects, or “bridges”, between monitor patch 20 and server 60. Bridge 40 communicates with monitor patch 20 over communication link 30 operating, in these exemplary embodiments, at approximately 915 MHz and at a power level that enables communication link 30 to function up to a distance of approximately 3 meters. It is preferable to place a bridge 40 in each room and at regular intervals along hallways of the healthcare facility where it is desired to provide the ability to communicate with monitor patches 20. Bridge 40 also is able to communicate with server 60 over network link 50 using any of a variety of computer communication systems including hardwired and wireless Ethernet using protocols such as 802.11a/big or 802.3af. As the communication protocols of communication link 30 and network link 50 may be very different, bridge 40 provides data buffering and protocol conversion to enable bidirectional signal transmission between monitor patch 20 and server 60.

While the embodiments illustrated by FIG. 1 employ a bridge 20 to provide communication link between the monitor patch 20 and the server 60, in certain alternative embodiments, the monitor patch 20 may engage in direct wireless communication with the server 60. In such alternative embodiments, the server 60 itself or a wireless modern connected to the server 60 may include a wireless communication system to receive data from the monitor patch 20.

In use, a monitor patch 20 is applied to a patient 10 by a clinician when it is desirable to continuously monitor basic vital signs of patient 10 while patient 10 is, in this embodiment, in a hospital. Monitor patch 20 is intended to remain attached to patient 10 for an extended period of time, for example, up to 5 days in certain embodiments, limited by the battery life of monitor patch 20. In some embodiments, monitor patch 20 is disposable when removed from patient 10.

Server 60 executes analytical protocols on the measurement data that it receives from monitor patch 20 and provides this information to clinicians through external workstations 100, preferably personal computers (PCs), over the hospital network 70. Server 60 may also send messages to mobile devices 90, such as cell phones or pagers, over a mobile device link 80 if a measurement signal exceeds specified parameters. Mobile device link 80 may include the hospital network 70 and internal or external wireless communication systems that are capable of sending messages that can be received by mobile devices 90.

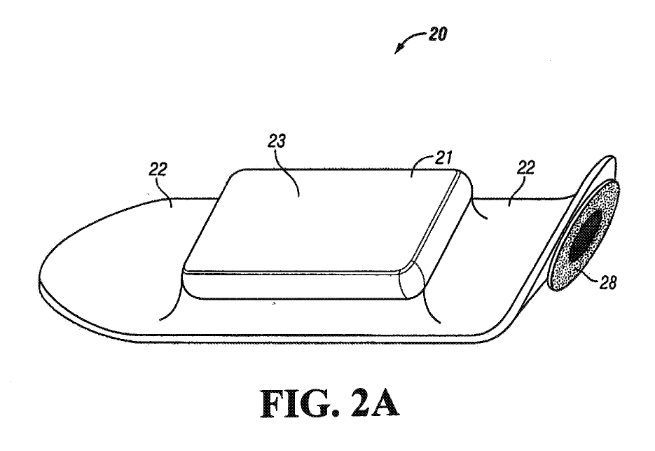

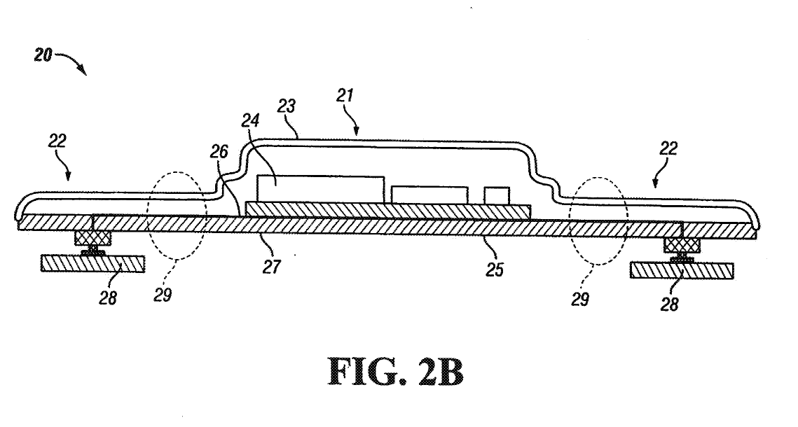

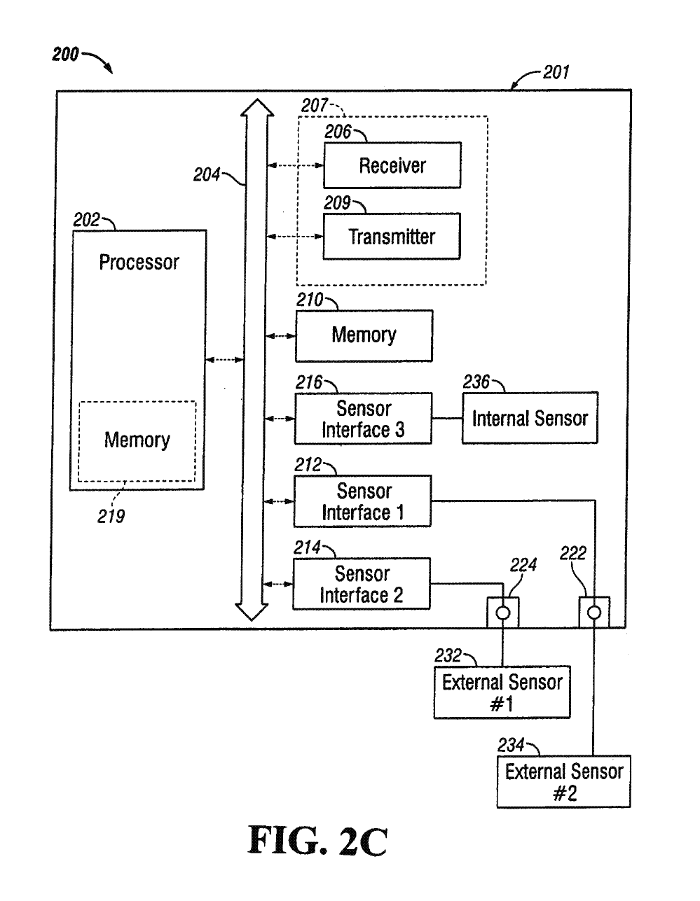

FIG. 2A is a perspective view of the vital-signs monitor patch 20 shown in FIG. 1 according to certain aspects of the present disclosure. In the illustrated embodiment, the monitor patch 20 includes component carrier 23 comprising a central segment 21 and side segments 22 on opposing sides of the central segment 21. In certain embodiments, the central segment 21 is substantially rigid and includes a circuit assembly (24, FIG. 2B) having electronic components and battery mounted to a rigid printed circuit board (PCB). The side segments 22 are flexible and include a flexible conductive circuit (26, FIG. 2B) that connect the circuit assembly 24 to electrodes 28 disposed at each end of the monitor patch 20, with side segment 22 on the right shown as being bent upwards for purposes of illustration to make one of the electrodes 28 visible in this view.

FIG. 2B is a cross-sectional view of the vital-signs patch 20 shown in FIGS. 1 and 2A according to certain aspects of the present disclosure. The circuit assembly 24 and flexible conductive circuit 26 described above can be seen herein. The flexible conductive circuit 26 operably connects the circuit assembly 24 to the electrodes 28. Top and bottom layers 23 and 27 form a housing 25 that encapsulate circuit assembly 28 to provide a water and particulate barrier as well as mechanical protection. There are sealing areas on layers 23 and 27 that encircles circuit assembly 28 and is visible in the cross-section view of FIG. 2B as areas 29. Layers 23 and 27 are sealed to each other in this area to form a substantially hermetic seal. Within the context of certain aspects of the present disclosure, the term ‘hermetic’ implies that the rate of transmission of moisture through the seal is substantially the same as through the material of the layers that are sealed to each other, and further implies that the size of particulates that can pass through the seal are below the size that can have a significant effect on circuit assembly 24. Flexible conductive circuit 26 passes through portions of sealing areas 29 and the seal between layers 23 and 27 is maintained by sealing of layers 23 and 27 to flexible circuit assembly 28. The layers 23 and 27 are thin and flexible, as is the flexible conductive circuit 26, allowing the side segment 22 of the monitor patch 20 between the electrodes 28 and the circuit assembly 24 to bend as shown in FIG. 2A.

FIG. 2C is a functional block diagram 200 illustrating exemplary electronic and sensor components of the monitor patch 20 of FIG. 1 according to certain aspects of the present disclosure. The block diagram 200 shows a processing and sensor interface module 201 and external sensors 232, 234 connected to the module 201. In the illustrated example, the module 201 includes a processor 202, a wireless transceiver 207 having a receiver 206 and a transmitter 209, a memory 210, a first sensor interface 212, a second sensor interface 214, a third sensor interface 216, and an internal sensor 236 connected to the third sensor interface 216. The first and second sensor interfaces 212 and 214 are connected to the first and second external sensors 232, 234 via first and second connection ports 222, 224, respectively. In certain embodiments, some or all of the aforementioned components of the module 201 and other components are mounted on a PCB.

Each of the sensor interfaces 212, 214, 216 can include one or more electronic components that are configured to generate an excitation signal or provide DC power for the sensor that the interface is connected to and/or to condition and digitize a sensor signal from the sensor. For example, the sensor interface can include a signal generator for generating an excitation signal or a voltage regulator for providing DC power to the sensor. The sensor interface can further include an amplifier for amplifying a sensor signal from the sensor and an analog-to-digital converter for digitizing the amplified sensor signal. The sensor interface can further include a filter (e.g., a low-pass or bandpass filter) for filtering out spurious noises (e.g., a 60 Hz noise pickup).

The processor 202 is configured to send and receive data (e.g., digitized signal or control data) to and from the sensor interfaces 212, 214, 216 via a bus 204, which can be one or more wire traces on the PCB. Although a bus communication topology is used in this embodiment, some or all communication between discrete components can also be implemented as direct links without departing from the scope of the present disclosure. For example, the processor 202 may send data representative of an excitation signal to the sensor excitation signal generator inside the sensor interface and receive data representative of the sensor signal from the sensor interface, over either a bus or direct data links between processor 202 and each of sensor interface 212, 214, and 216.

The processor 202 is also capable of communication with the receiver 206 and the transmitter 209 of the wireless transceiver 207 via the bus 204. For example, the processor 202 using the transmitter and receiver 209, 206 can transmit and receive data to and from the bridge 40. In certain embodiments, the transmitter 209 includes one or more of an RF signal generator (e.g., an oscillator), a modulator (a mixer), and a transmitting antenna; and the receiver 206 includes a demodulator (a mixer) and a receiving antenna which may or may not be the same as the transmitting antenna. In some embodiments, the transmitter 209 may include a digital-to-analog converter configured to receive data from the processor 202 and to generate a base signal; and/or the receiver 206 may include an analog-to-digital converter configured to digitize a demodulated base signal and output a stream of digitized data to the processor 202.

The processor 202 may include a general-purpose processor or a specific-purpose processor for executing instructions and may further include a memory 219, such as a volatile or non-volatile memory, for storing data and/or instructions for software programs. The instructions, which may be stored in a memory 219 and/or 210, may be executed by the processor 202 to control and manage the wireless transceiver 207, the sensor interfaces 212, 214, 216, as well as provide other communication and processing functions.

The processor 202 may be a general-purpose microprocessor, a microcontroller, a Digital Signal Processor (DSP), an Application Specific Integrated Circuit (ASIC), a Field Programmable Gate Array (FPGA), a Programmable Logic Device (PLD), a controller, a state machine, gated logic, discrete hardware components, or any other suitable device or a combination of devices that can perform calculations or other manipulations of information.

Information, such as program instructions, data representative of sensor readings, preset alarm conditions, threshold limits, may be stored in a computer or processor readable medium such as a memory internal to the processor 202 (e.g., the memory 219) or a memory external to the processor 202 (e.g., the memory 210), such as a Random Access Memory (RAM), a flash memory, a Read Only Memory (ROM), a Programmable Read-Only Memory (PROM), an Erasable PROM (EPROM), registers, a hard disk, a removable disk, or any other suitable storage device.

In certain embodiments, the internal sensor 236 can be one or more sensors configured to measure certain properties of the processing and sensor interface module 201, such as a board temperature sensor thermally coupled to a PCB. In other embodiments, the internal sensor 236 can be one or more sensors configured to measure certain properties of the patient 10, such as a motion sensor (e.g., an accelerometer) for measuring the patient's motion.

The external sensors 232, 234 can include sensors and sensing arrangements that are configured to produce a signal representative of one or more vital signs of the patient to which the monitor patch 20 is attached. For example, the first external sensor 232 can be a set of sensing electrodes that are affixed to an exterior surface of the monitor patch 20 and configured to be in contact with the patient for measuring the patient's respiratory rate, and the second external sensor 234 can include a temperature sensing element (e.g., a thermocouple or a thermistor) affixed, either directly or via an interposing layer, to skin of the patient 10 for measuring the patient's body temperature. In other embodiments, one or more of the external sensors 232, 234 or one or more additional external sensors can measure other vital signs of the patient, such as blood pressure and pulse rate.

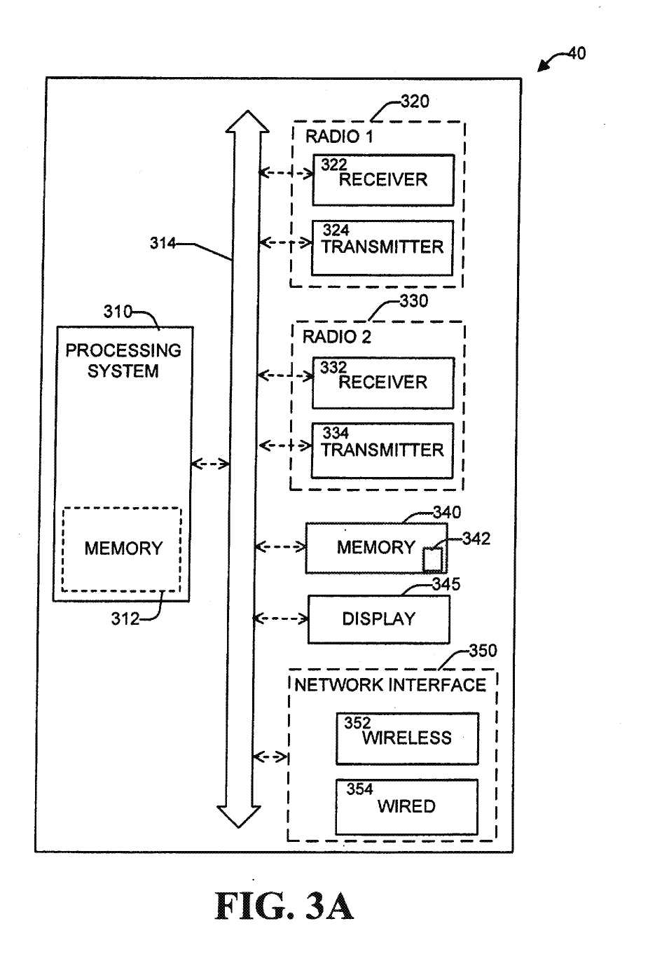

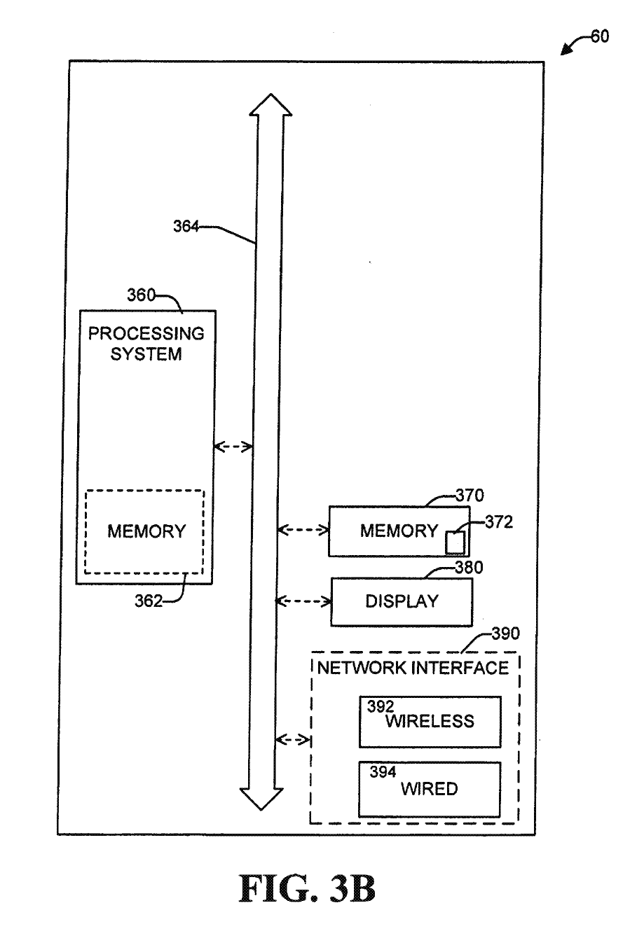

FIG. 3A is a functional block diagram illustrating exemplary electronic components of bridge 40 of FIG. 1 according to one aspect of the subject disclosure. Bridge 40 includes a processor 310, radio 320 having a receiver 322 and a transmitter 324, radio 330 having a receiver 332 and a transmitter 334, memory 340, display 345, and network interface 350 having a wireless interface 352 and a wired interface 354. In some embodiments, some or all of the aforementioned components of module 300 may be integrated into single devices or mounted on PCBs.

Processor 310 is configured to send data to and receive data from receiver 322 and transmitter 324 of radio 320, receiver 332 and transmitter 334 of radio 330 and wireless interface 352 and wired interface 354 of network interface 350 via bus 314. In certain embodiments, transmitters 324 and 334 may include a radio frequency signal generator (oscillator), a modulator, and a transmitting antenna, and the receivers 322 and 332 may include a demodulator and antenna which may or may not be the same as the transmitting antenna of the radio. In some embodiments, transmitters 324 and 334 may include a digital-to-analog converter configured to convert data received from processor 310 and to generate a base signal, while receivers 322 and 332 may include analog-to-digital converters configured to convert a demodulated base signal and sent a digitized data stream to processor 310.

Processor 310 may include a general-purpose processor or a specific-purpose processor for executing instructions and may further include a memory 312, such as a volatile or non-volatile memory, for storing data and/or instructions for software programs. The instructions, which may be stored in memories 312 or 340, may be executed by the processor 310 to control and manage the transceivers 320, 330, and 350 as well as provide other communication and processing functions.

Processor 310 may be a general-purpose microprocessor, a microcontroller, a Digital Signal Processor (DSP), an Application Specific Integrated Circuit (ASIC), a Field Programmable Gate Array (FPGA), a Programmable Logic Device (PLD), a controller, a state machine, gated logic, discrete hardware components, or any other suitable device or a combination of devices that can perform calculations or other manipulations of information.

Information such as data representative of sensor readings may be stored in memory 312 internal to processor 310 or in memory 340 external to processor 310 which may be a Random Access Memory (RAM), flash memory, Read Only Memory (ROM), Programmable Read Only Memory (PROM), Erasable Programmable Read Only Memory (EPROM), registers, a hard disk, a removable disk, a Solid State Memory (SSD), or any other suitable storage device.

Memory 312 or 340 can also store a list or a database of established communication links and their corresponding characteristics (e.g., signal levels) between the bridge 40 and its related monitor patches 20. In the illustrated example of FIG. 3A, the memory 340 external to the processor 310 includes such a database 342; alternatively, the memory 312 internal to the processor 310 may include such a database.

FIG. 3B is a functional block diagram illustrating exemplary electronic components of server 60 of FIG. 1 according to one aspect of the subject disclosure. Server 60 includes a processor 360, memory 370, display 380, and network interface 390 having a wireless interface 392 and a wired interface 394. Processor 360 may include a general-purpose processor or a specific-purpose processor for executing instructions and may further include a memory 362, such as a volatile or non-volatile memory, for storing data and/or instructions for software programs. The instructions, which may be stored in memories 362 or 370, may be executed by the processor 360 to control and manage the wireless and wired network interfaces 392, 394 as well as provide other communication and processing functions.

Processor 360 may be a general-purpose microprocessor, a microcontroller, a Digital Signal Processor (DSP), an Application Specific Integrated Circuit (ASIC), a Field Programmable Gate Array (FPGA), a Programmable Logic Device (PLD), a controller, a state machine, gated logic, discrete hardware components, or any other suitable device or a combination of devices that can perform calculations or other manipulations of information.

Information such as data representative of sensor readings may be stored in memory 362 internal to processor 360 or in memory 370 external to processor 360 which may be a Random Access Memory (RAM), flash memory, Read Only Memory (ROM), Programmable Read Only Memory (PROM), Erasable Programmable Read Only Memory (EPROM), registers, a hard disk, a removable disk, a Solid State Memory (SSD), or any other suitable storage device.

Memory 362 or 370 can also store a database of communication links and their corresponding characteristics (e.g., signal levels) between monitor patches 20 and bridges 40. In the illustrated example of FIG. 3B, the memory 370 external to the processor 360 includes such a database 372; alternatively, the memory 362 internal to the processor 360 may include such a database.

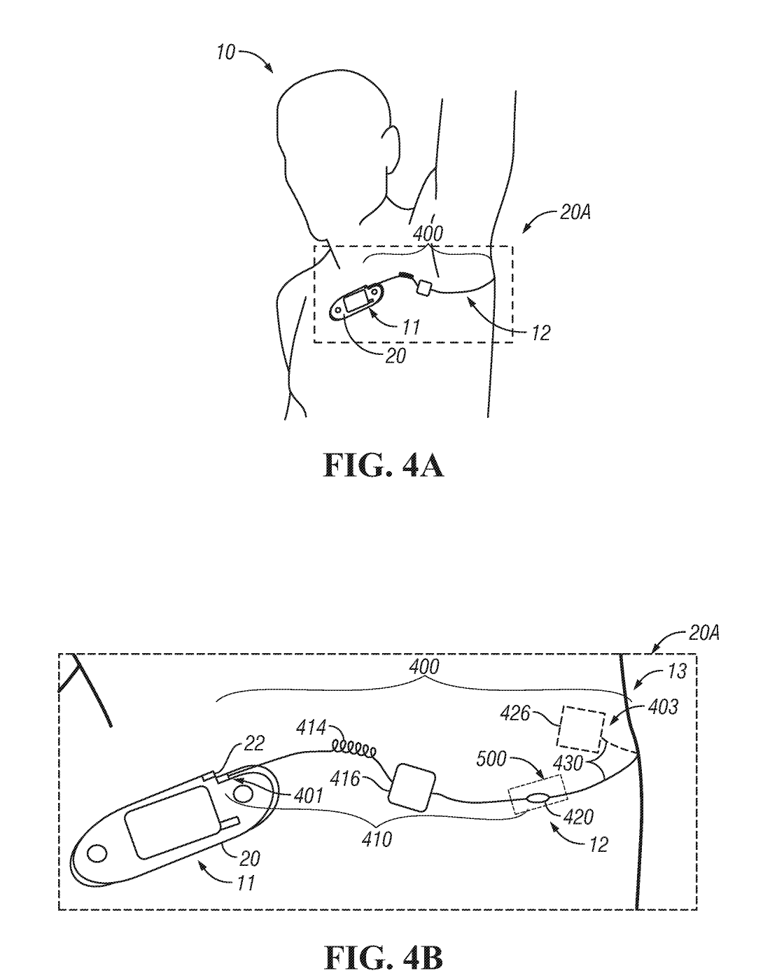

As indicated above with respect to FIG. 2C, certain embodiments of the monitor patch 20 are configured to operate with external sensors that are in turn configured to produce a signal representative of one or more vital signs of the patient to whom the monitor patch 20 is attached. For example, the second external sensor 234 can be a temperature probe that includes a temperature sensing element (e.g., a thermocouple or thermistor) affixed, either directly or via an interposing layer, to skin of the patient 10 for measuring the patient's body temperature. FIG. 4A is a diagram depicting a patient 10 wearing a temperature monitoring system 20A comprising a monitor patch 20 and a temperature probe 400 that is configured to measure body temperature of the patient 10. In the illustrated example, the temperature probe 400 is configured for axillary temperature sensing of the patient 10 to whom the monitor patch 20 is attached. The monitor patch 20 is attached to the chest 11 of the patient 10, with a sensing portion of the temperature probe 400 retained in the axilla 12 of the patient 10 during body temperature monitoring.

FIG. 4B is a diagram providing an enlarged view of the monitoring system 20A depicted in FIG. 4A according to certain aspects of the present disclosure. As indicated above, the monitor patch 20 is attached to the chest 11 of the patient 10 via, e.g., an adhesive backing (not shown). The temperature probe 400 has a proximal end 401 and a distal end 403 and includes a wiring portion 410, a body connection portion 430, and a sensing portion 420 disposed between the wiring and body connection portions 410, 430. The proximal end 401 of the temperature probe 400 is connected to the monitor patch 20 at its connection port 22. In certain embodiments, the proximal end 401 of the temperature probe 400 is removably attached (e.g., plugged) to the monitor patch 20. In other embodiments, the proximal end 401 is fixedly attached (e.g., epoxied or fused) to the monitor patch 20.

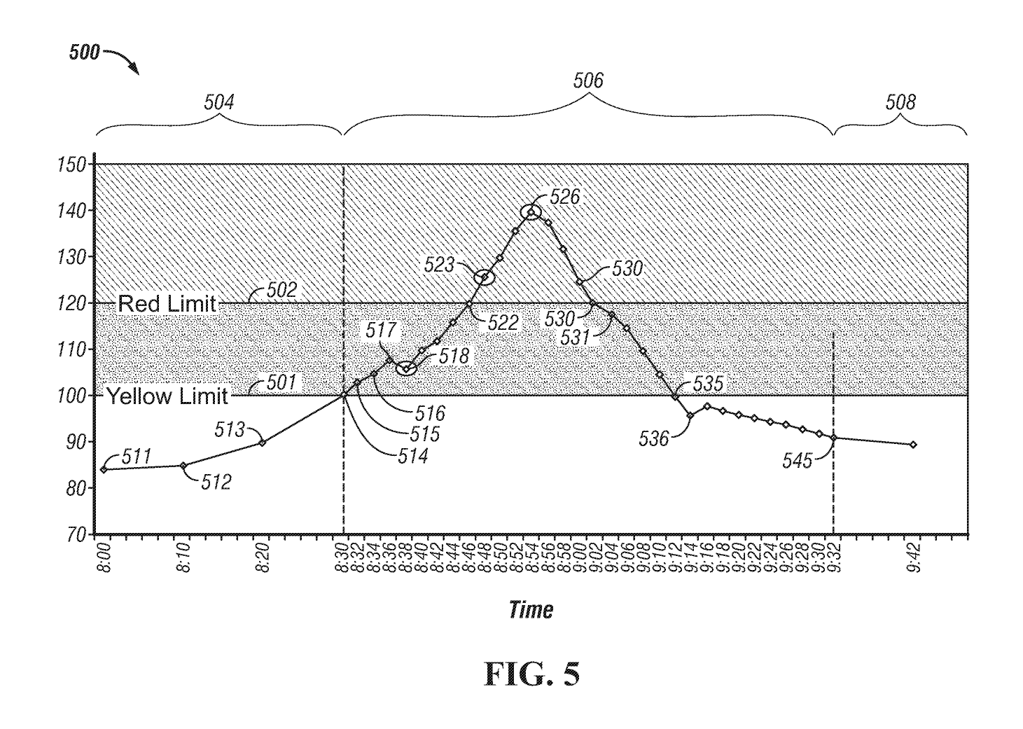

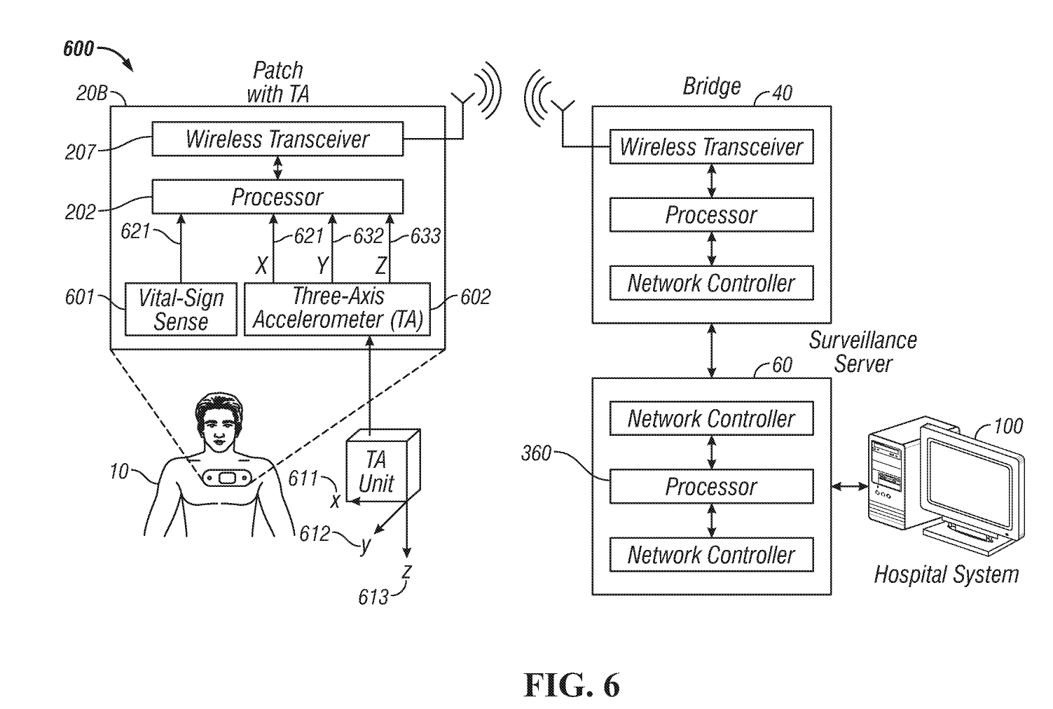

The sensing portion 420 of the temperature probe 400 is configured for placement within the axilla 12 of the patient 10 and includes a temperature sensing element (e.g., 234A-D of FIGS. 5A-D and 234E-F of FIGS. 6A-B). The wiring portion 410 of the temperature probe 400 includes one or more electrical conductors (512, 514 of FIGS. 5A-D and FIGS. 6A-B) for carrying a signal responsive to a change in body temperature of the patient 10 between the temperature sensing element 234 and the monitor patch 20. In the illustrated example, the wiring portion 410 includes a flexible cable comprising a tubing and electrical conductors (e.g., a pair of twisted copper wires) placed within the tubing. The wiring portion 410 includes a coiled section 414 acting as a spring to take up any slack in the cable so as to accommodate patients of different sizes. In the illustrated example, the monitoring system 20A further includes an adhesive element 416 (e.g., a tape) coupled to the cable and configured to attach the wiring portion 410 of the cable to the patient's body, e.g., at a point between the chest 11 and the armpit 12 of the patient.

The body connection portion 430 has one end connected to the sensing portion 420 and is configured to be attached to another body portion of the patient 10 such that the sensing portion 420 of the temperature probe 400 can be retained within the axilla 12 of the patient 10. In the illustrated example, such attachment is achieved via an adhesive element 426 (e.g., a tape) coupled to the distal end of the body connection portion 430. The coupled adhesive element 426 is then attached to a second body portion 13 (e.g., the back of the patient's arm) of the patient 10.

A multitude of modifications and additions to the illustrated embodiment of FIG. 4B are possible without departing from the scope of the disclosure. For example, the body connection portion 430 of the temperature probe 400 can include one or more coiled sections acting as a spring similar to the coiled section 414 of the wiring portion 410. The adhesive element 416 may be coupled to the body connection portion 430 at a point different than the distal end of the body connection portion 430. In certain embodiments, entirely different means of attaching the body connection portion 430 to the patient's body may be used. For example, the body connection portion 430 may itself be in the form of an adhesive tape that can stick to the body of the patient 10 or may include an elastic loop (e.g., a rubber band) to be placed around the patient's arm.

While the temperature probe 400 in the illustrated embodiments of FIGS. 4A-B is shown to be operatively coupled to a vital-sign monitor patch worn by the patient 10, the temperature probe 400 may be alternatively operatively coupled to other types of monitoring devices such as a stationary monitoring unit located near the patient's hospital bed. Such a stationary monitoring unit can take readings of the patient's body temperature based on a signal from the temperature probe 400 and send the temperature readings to a surveillance server via a wired or wireless connection and make other decisions such as providing an indication of an alarm condition (e.g., a high body temperature condition or a loss of thermal contact between the temperature probe and the patient).

As indicated above, in certain aspects, the vital-signs monitoring system (e.g., the monitoring system 12 of FIG. 1) of the present disclosure can be configured to sound an alarm when one or more vital-sign readings exceed an alarm threshold limit. For example, the surveillance server 60 can receive vital-sign readings (e.g., body temperature values or heart rate values) from the monitor patch 20, either directly or via the bridge 40, and, if the vital-sign readings satisfy an alarm condition, e.g., by exceeding an alarm threshold limit for a certain predetermined number of samples, send an alarm notification to the hospital system 100.

FIG. 5 depicts a graph 500 representing a series of data samples (solid dots) corresponding to vital-sign readings of a patient generated by a monitor patch 20 according to certain aspects of the present disclosure. The vital-sign readings can represent, for example, body temperature or heart rate of the patient. The graph 500 shows two threshold limits: a first threshold limit 501 (the “yellow limit”) and a second threshold limit 502 (the “red limit”). The first threshold limit 501 can correspond to an alert or warning threshold limit, whereas the second threshold limit 502 can correspond to an alarm threshold limit. The exemplary graph 500 is divided into three regions: a first region 504, a second region 506, and a third region 508. In the first region 504, the vital-sign readings are below the first threshold limit 501, and the monitoring system is in its normal mode of operation. In the normal mode of operation, vital-sign readings 511-513 are generated at the monitor patch 20 and sent to the surveillance server 60 at a first (slow) sampling rate. The surveillance server 60 stores the received vital-sign readings 511-513 in a database and/or forwards them to a hospital system (e.g., workstation 100 of FIG. 1) to be displayed at the first sampling rate.

In the second region 506, entered after the vital-sign readings exceed the first (alert) threshold limit 501, the monitoring system is in an alert mode of operation. In the alert mode of operation, vital-sign readings 514-545 are generated at the monitor patch 20 and sent to the surveillance server 60 at a second (fast) rate. The surveillance server 60 can store the received vital-sign readings 514-545 to a database and/or forward them to the hospital system 100 to be displayed at the second sampling rate. In addition, the surveillance server 60 can also send alarm notifications to the hospital system 100 when the vital-sign reading exceed the second (alarm) threshold limit 502 and stay above the limit for a predetermined number of samples. Such alarm notifications may be sent each time the reading exceeds the second threshold limit 502 or after every M consecutive times the readings stay above the second threshold limit 502, where M is an integer greater than 1.

In the third region 508, entered after the vital-sign readings have fallen and stayed below the first threshold limit 501 for N number of times, where N is a positive integer (e.g., 1-4), the monitoring system has reverted back to the normal mode of operation, where vital-sign readings are generated at the monitor patch 20 and sent to the surveillance server 60 at the first (slow) sampling rate again.

For a number of reasons, a vital sign monitoring system may sound so called “false” or “nuisance” alarms when such alarms are not warranted. Under certain circumstances, the system may sound false alarms so often and in situations that are not truly serious that such alarms may start to be ignored by clinicians, thereby creating the “cry wolf” syndrome where an alarm caused by a true emergency situation may be also ignored. One source of false alarms is motion of a patient to whom the monitor, patch 20 is attached. It is known that motion associated with certain types of physical activity by the patient can lead to variations in vital-sign readings. For example, a brisk walking by the patient, e.g., down the hall or on a treadmill, can cause an increase in the heart rate and respiratory rate of the patient, and the rates can eventually exceed respective alarm threshold limits, thereby setting off alarms. However, in most cases, such a temporary motion-related increase in a vital-sign value has no clinical significance and may be safely ignored.

Motion of a patient can cause a false alarm not only through an increase in a vital-sign reading but also through so-called “motion artifacts” that the motion can generate. For example, a twisting or stretching motion, e.g., during a vigorous exercise by the patient, can cause changes in the spacing between the respiratory-rate monitoring electrodes in the monitor patch 20, and such changes in the inter-electrode spacing can produce spurious spikes in the measured respiratory-rate readings. These same movements can also generate forces on the electrodes. The electrode-skin electrical interface is very sensitive to any force being applied in that area, either shear, tension or compression forces relative to skin-electrode plane. This can have a significant impact on the electrical signal for both ECG and impedance pneumography.

Furthermore, position (postural orientation) of the patient can also affect the measured vital-sign readings. For example, a patient lying on his or her side or in a prone position on the bed can put a stress or strain on a sensing element of the monitor patch, and the stress or strain can cause a vital-sign reading to increase beyond a high alarm threshold limit set or decrease below a low alarm threshold limit. Motional or positional disruptions can also cause false positives, as mentioned above, but also false negatives. Signal can be disrupted in such a way that the reading appears normal while actual vital signs call for critical medical attentions. For example, a reading could be affected by the patient movement in such a way that the false value being sent falls into a safe domain while the actual vital sign value is outside the safe domain. In this case, no alarm is sent while the condition of the patient requires it.

In addition, random movements (e.g. dressing/undressing, rolling in a bed) has a tendency to diminish the quality of the signal, leading to potentially less accurate values. Cyclic movements (walking, combing hair, turning pages of a book) would have the effect of creating changes in impedance which could be mistaken for breaths. This can lead to a wrong value being sent. This second situation is particularly difficult to identify as the impedance patterns could look very similar to those related to respiration, for example.

If motion and/or position (motion/position) of the patient wearing the monitor patch 20 can be determined, however, false alarms or false negatives due to motion/position artifacts or clinically-insignificant temporary motion/position-related changes to vital-sign readings may be prevented or at least reduced, for example, by recognizing and ignoring samples plagued by motion/position-related artifacts or adjusting one or more alarm threshold limits accordingly.

FIG. 6 is a diagram depicting an exemplary vital-sign monitoring system 600 that is configured to determine motion and/or position of a patient (hereinafter the “patient motion/position”) and thereby prevent or reduce false alarms associated with the patient motion/position according to certain aspects of the present disclosure. The system 600 includes a monitor patch 20B, a bridge 40, a surveillance server 60, and a hospital system 100. The monitor patch 20B includes a vital-sign sensing module 601, an accelerometer 602, a processor 202, and a wireless transceiver 207. With reference to FIG. 2C, the vital-sign sensing module 602 can include one or more vital-sign sensors 232, 234, such as electrodes for measuring respiration and temperature sensing elements, and sensor interfaces 212, 214 for conditioning and converting signals from the sensors 332, 234. The vital-sign sensing module 601 provides one or more vital-sign outputs 621 responsive to changes in one or more vital signs of the patient 10.

In certain embodiments, the accelerometer 602 is a three-axis accelerometer (TA) configured to provide three outputs 621, 622, 623 responsive to accelerations in three orthogonal directions, e.g., x, y, and z-directions 611, 612, and 613. The outputs 621, 622, 623 may be analog or digital signals. An example of a three-axis accelerometer that can be employed is a three-axis linear accelerometer (Model No. LIS302DL) manufactured by STMicroelectronics, which provides selectable full scales of +/−2 g and +/−8 g. In the illustrated example, the x-direction 611 is along the length direction of the monitor patch 20B; the y-direction 612 is perpendicular to the upper surface of the monitor patch 20B, and the z-direction 613 is along the width direction of the monitor patch 20B. The TA 602 produces three outputs: x output 631, y output 632, and z output 633, corresponding to measured accelerations in the x-, y- and z-directions 611, 612, 613, respectively.

In certain embodiments of the monitor patch 20B, the processor 202 is configured (e.g., programmed) to receive the one or more vital-sign outputs 621 from the vital-sign sensing module 601 and the x, y, z outputs 631, 632, 633 from the TA 602 and determine (e.g., calculate or look up) vital-sign readings and x-, y-, and z-acceleration readings from the received vital-sign and acceleration outputs. Given the geometry of the illustrated example, the x-acceleration reading is indicative of the acceleration that the patient 10 is subjected to along the width (e.g., side-to-side) direction of the patient 10, the y-acceleration reading is indicative of the acceleration that the patient 10 is subjected to along the thickness (e.g., front-to-back) direction of the patient 10, and the z-acceleration reading is indicative of the acceleration the patient 10 is subjected to along the height (e.g., head-to-toe) direction of the patient 10. For example, if the patient 10 were standing up still as in the illustrated example of FIG. 6, the x-, y-, z-acceleration readings, normalized to the Earth's gravitational acceleration (g), would be (0, 0, 1). If the patient 10 were lying in a prone (facing down) position or in a supine (facing up) position, the normalized acceleration readings would be (0, −1, 0). The processor 202 of the monitor patch 20B is further configured to send the vital-sign readings and the x-, y-, and z-acceleration readings to the surveillance server 60, either directly or via bridge 40.

At the surveillance server 60, a processor (e.g., 360 of FIG. 3B or 202 of FIG. 6) is configured (e.g., programmed) to execute a program stored in a memory (either inside or outside the processor 360, 202) and to receive the vital-sign and acceleration readings from the monitor patch 20B. The processor 360, 202 is further configured to determine one or both of motion and position of the patient 10 (patient motion/position) after applying one or more motion/position determination algorithms to the acceleration readings. The processor 360, 202 is further configured to modify an alarm condition determination procedure if the motion/position determination satisfies a predefined condition. The modification to the alarm condition determination procedure can include adjusting one or more alarm threshold limits to account for the likely outcome that the patient motion/position would cause changes in the vital-sign readings, where the changes (e.g., increases) do not have any clinical significance for the patent. The modification can further exclude one or more vital-sign reading from the alarm condition determination procedure if it is determined that the patient motion/position is likely to cause artifacts (e.g., spikes) in the readings or generate a band reading movement because, for example, the patient movement can be mistaken for respiration. In certain embodiments, acceleration readings can also be used to provide input to a noise filtering algorithm allowing for a better filtering of cyclic movements. For example, knowing the frequency of the gait of a patient who is walking could allow excluding that particular harmonic from an impedance signal.

Regarding the patient's position (e.g., postural orientation), the processor 360 can determine whether the patient is in a prone, sitting, or standing position, e.g., from the relative values of the x-, y-, and z-acceleration readings. In some embodiments, if the processor 360 determines that the patient 10 is in a prone position, the processor 360 further determines whether the patient 10 is lying in a supine or prone position on his/her front, back or side (right or left).

Regarding the patent's motion, the processor 360 can determine whether the patient is in engaged in motion or is at rest from the x-, y-, and z-acceleration readings. One suitable measure of such motion/rest determination can be the normal signal magnitude area (SMA). Defined in (1), the SMA can be used as the basis for identifying periods of motion or activity:

$$SMA = \frac{1}{t} ( {\int }_{0}^{t} \vert x ( t ) \vert d t + {\int }_{0}^{t} \vert y ( t ) \vert d t + {\int }_{0}^{t} \vert z ( t ) \vert d t ) \qquad ( 1 )$$

where x(t), y(t), and z(t) refer to the body components of the x-, y-, and z-accelerations, respectively. By way of example, calculation of this parameter can be performed by summing each sampled value progressively (e.g., following the digitization of each accelerometer sample) over a 1-s interval. The SMA value is compared to an appropriate activity threshold value, which can be theoretically or experimentally determined. If the SMA value is at or above the activity threshold value, the patient 10 is deemed to be engaged in a motion, whereas, if the SMA value is below the activity threshold value, the patient 10 is deemed to be at rest.

When the processor 360 determines that the patient 10 is engaged in motion, the processor 360 can further determine whether the patient's motion is active or passive, meaning whether the patient 10 is moving (e.g., ambulating) or is being moved (e.g., on a patient carrier or a motorized wheelchair). This determination can be performed by analyzing a combination of motion and position information derived from the acceleration readings. For example, if the acceleration readings indicate that the patient 10 is moving along the z-direction, while the y-direction acceleration indicates that the patient 10 is lying down in a prone position, the processor 360 can determine that the patient 10 is being moved while lying down on a patient carrier and, accordingly, determine that the patient's motion is passive. If the processor 360 determines that the patient's motion is passive, the processor 360 applies a standard alarm condition determination procedure, an example of which was described above with respect to FIG. 5, without any modification to the procedure.

On the other hand, if the processor 360 determines that the patient's motion is active and that the patient's active motion is of the type and intensity that is likely to cause one or more vital-sign readings (e.g., heart rate) of the patient 10 to exceed the currently active standard alarm threshold limit and trigger an alarm without having any clinical significance to the patient, the processor 360 makes a modification to the alarm condition determination procedure, e.g., by increasing the alarm threshold limit (e.g., the second threshold limit 502 of FIG. 5). In certain embodiments, the amount of increase for the alarm threshold limit can fixed (e.g., 10%). In other embodiments, the amount of increase is linearly proportional to or is another increasing function that depends on the level of a patient's physical activity as determined by the acceleration readings and a total duration of the physical activity. In yet other embodiments, the motion is classified into different activity categories, and the amount of increase is determined based on the classified activity category. For example, the patent's active ambulatory motion may be classified into one of: 1) slow walking; 2) normal walking; 3) fast walking; and 4) running. Depending on the activity category for the patient's motion (and in some cases, duration thereof), the processor 360 can increase the alarm threshold limit by different amounts.

In some embodiments, the processor 360 determines that a change in the patient motion/position has occurred. For example, the processor 360 may determine that the patient 10 has changed from sitting to standing positions (sit-to-stand), from standing to sitting positions (stand-to-sit), prone to sit positions (prone-to-sit), or sit to prone positions (sit-to-prone). Such positional change determination may involve the processor 360 analyzing changes in the acceleration readings and finding certain known patterns that signify one of the positional changes listed above. Such positional change information may be used as time-markers to start a clock. For example, when a patient stands up from a bench and starts to walk briskly down a hall, the sit-to-stand change can start a timer for measuring the duration of the physical activity, which duration can be used for calculating the amount of increase for the alarm threshold limit.

In some embodiments, if the processor 360 determines that the patient 10 is engaged in a type of motion (e.g., running) and/or is in a position (e.g., lying on the front, back or side) that is likely to produce motion/position-related artifacts or difficulties for the system to acquire an accurate reading when the patient maintains a specific position (e.g., by putting stress or strain on a sensing element of the patch 20B or by generating changes in impedance which are not related to respiration), the processor 360 excludes all or some of the received vital-sign readings from the alarm condition determination procedure or refrains from sending an alarm notification to hospital system 100 even if the vital-sign readings exceed the alarm threshold limit.

In certain embodiments, the processor 360 may perform a check across readings for different vital-signs signs to rule out possible false positives in the patient motion/position determination. For example, assume that the motion/position determination procedure indicates that the patient 10 is engaged in a type of activity (e.g., running) that is likely to substantially increase both the heart rate and the respiratory rate of the patient 10. If the vital-sign readings indicate, however, that only the respiratory rate, but not the heart rate, has increased, the processor 360 may determine that the increase in the heart rate is not motion-related and decide not to modify the alarm determination procedure (e.g., increase the alarm threshold limit) for the heart rate. By performing such a cross checking based on different vital-sign readings, the motion/position determination procedure itself may be verified, thereby increasing the reliability of the procedure for preventing or reducing false alarms.

It shall be appreciated that a multitude of modifications to the patient monitoring system 600 described above are possible without departing from the scope of the present disclosure. For example, while the accelerometer 602 in the exemplary embodiment of FIG. 6 provides outputs 631, 632, 633 in three Cartesian-coordinate directions (e.g., x, y, and z), in alternative embodiments, the accelerometer 602 may provide only one or two outputs (e.g., along z only or in x and y). In other alternative embodiments, the accelerometer 602 may provide three outputs in three cylindrical-coordinate directions (e.g., r, θ, and z) or spherical-coordinate directions (e.g., r, θ, and φ). While the accelerometer 602 is part of the monitor patch 20B in the exemplary embodiment of FIG. 6, in alternative embodiments, the accelerometer may be detached from the monitor patch 20B and communicate one or more acceleration outputs to the monitor patch 20B via wired or wireless connections.

In some embodiments, the motion/position determination procedure may be performed by the processor 202 in the motion patch 20B instead of the processor 360 in the surveillance server 60. In those embodiments, the processor 202, upon determining a patient motion/position that requires a modification to the alarm condition determination procedure, may send a message indicative of such motion/position determination to the surveillance server 60. In addition, the processor 202 of the monitor patch 20B, upon determining that the patient 10 is engaged in a motion or in a position that is likely to produce artifacts in vital-sign readings, may choose not to send the vital-sign readings while such a patient motion/position persists.

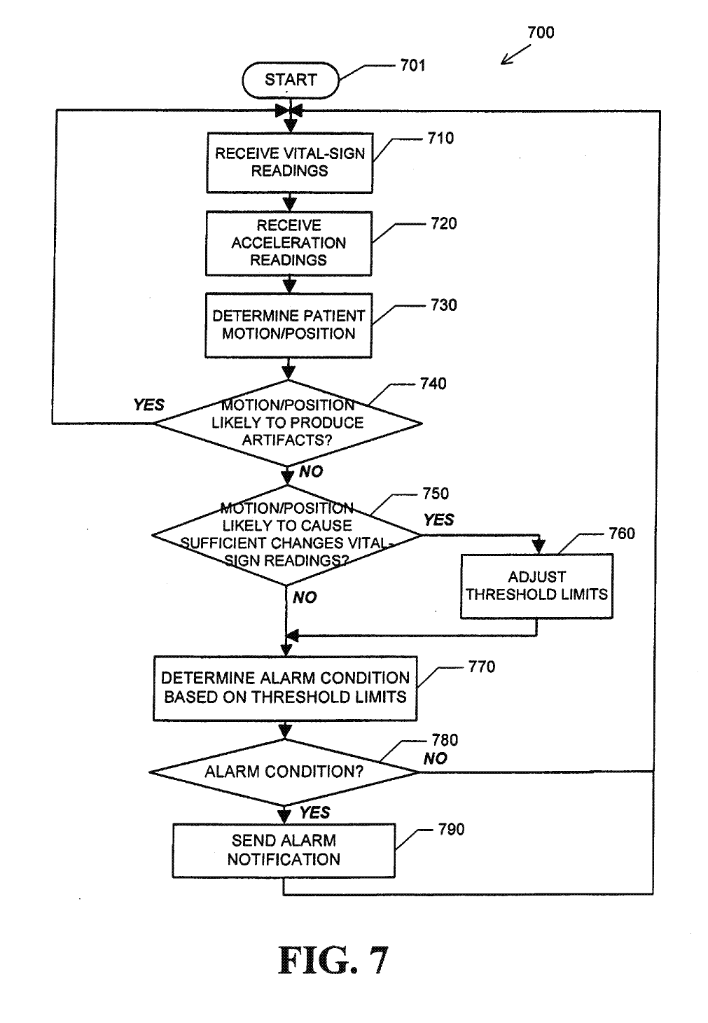

In certain embodiments, the acceleration readings can be used to search for acceleration patterns which are typically associated with respiration (e.g. cyclic accelerations along an horizontal axis pointing in front of the patient). This approach, as well the other methods of filtering some harmonics (described in a comment above), can generate a completely different process flow than the one presented in FIG. 7. In those two cases, acceleration readings can be used to help improve signal processing, as opposed to other methods described here where they are use only to improve the way vital sign readings are generating alarms.

FIG. 7 is a flowchart illustrating an exemplary process 700 for determining motion or position of a patient and thereby preventing or reducing false alarms associated with the patient motion/position according to certain aspects of the present disclosure. For ease of illustration, without any intent to limit the scope of the present disclosure in any way, the process 700 will be described with reference to the exemplary patient monitoring system 600 of FIG. 6. The process 700 begins at start state 701 and proceeds to operations 710 and 720 in which new sets of vital-sign readings and acceleration readings generated at and sent from the monitor patch 20B are received by the surveillance server 60. While the reception of vital-sign readings (operation 710) is shown as being performed prior to the reception of acceleration readings (operation 720) in the exemplary process 700 of FIG. 7, in alternative processes, the reception of acceleration readings by the surveillance server 60 may take place prior to or at the same time as the reception of the vital-sign readings. The frequency of the reception of acceleration readings may be the same or different from the frequency of the reception of the vital-sign readings. For example, the vital-sign readings may be received every 10 minutes while the acceleration readings may be received every 30 seconds during the normal mode of operation for the monitoring system. A high-frequency reception of the acceleration readings allows for determination of a position change (e.g., sit-to-stand) of the patient, for example.

The process 700 proceeds to operation 730 in which the patient motion/position is determined based at least in part on the received acceleration readings. Various types of motions and/or positions that may be so determined and methods by which such determination may be made are described above with respect to FIG. 6 and are not repeated here.

The process 700 proceeds to decision state 740 in which it is determined whether the patient motion/position determined at the operation 730 is likely to produce motion/position-related artifacts. Examples of such motions and positions that are likely to produce artifacts include, but are not limited to: twisting, stretching, lying in a prone position on the front or side, dressing/undressing, rolling in a bed, and showering. If the answer to the determination at the decision state 740 is YES (the patient motion/position likely to produce artifacts), the process 700 loops back to the operations 710 and 720 where new sets of vital-sign readings and acceleration readings are received. In the illustrated exemplary process, previously received vital-sign readings are excluded from an alarm condition determination procedure of operation 770 to be described below, and, hence, a false alarm associated with the motion/position-related artifacts is prevented.

On the other hand, if the answer to the determination at the decision state 740 is NO (the patient motion/position not likely to produce artifacts), the process 700 proceeds to another decision state 750 in which it is determined whether the patent motion/position determined at the operation 730 is likely to cause significant changes to vital-sign readings to exceed an alarm threshold limit so as to warrant a modification to the alarm condition determination procedure This determination can involve the processor 360 analyzing the acceleration readings and classifying the patient motion/position to one of several categories of motions and positions. For example, by comparing the acceleration readings to certain known patterns stored in a memory of the surveillance server 60 or in a memory accessible by the server 60 or the patch 20B, the processor 360 of the server 60 or the processor 202 of the patch 20B can categorize the patient motion/position into one of: slow walking, normal walking, fast walking, running, twisting, and jumping. Some non-limiting examples of position categories include: prone, sitting, standing, sit-to-stand, stand-to-sit, prone-to-sit, or sit-to-prone).

In some embodiments, in conjunction with determining whether the patient motion/position is likely to cause changes in one or more vital-sign readings significant enough to cause an alarm, it is determined whether the significant changes are likely to have a clinical significance for the patient. If it is determined that the patient motion/position, while causing such significant changes, nevertheless is not likely to have any clinical significance for the patient (e.g., a 20-year old male patient who recently had a tonsillectomy walking vigorously down a hall), the answer to the determination of the decision state 740 is YES. The same or a similar physical activity can have a different outcome depending on patient-specific information such as age, sex, and medical condition of the patient. For example, if the patient is a 70-year old female patient who recently suffered a heart attack, and she is walking vigorously in the hallway, the surveillance server 60, based on a rule database stored therein, for example, may determine that the activity is likely to cause significant changes in one or more vital-sign reading to activate an alarm, and such changes can have a clinical significance for the patient; and, accordingly, determine that the answer to the determination of the decision state 740 is NO. Therefore, in certain embodiments, the procedure or algorithm for the decision state 740 can take account of patient specific information such as age, sex, and medical conditions (present and past) of the patient 10.

If the answer to the determination at the decision state 740 is NO (e.g., the patient motion/position not likely to cause significant changes, or changes, even if significant, are likely to have a clinical significance), the process 700 proceeds to operation 770 in which a standard (unmodified) alarm condition determination procedure is applied to the vital-sign readings to determine whether to send an alarm notification to the hospital system 100. The alarm condition determination can involve comparing the vital-sign readings against one or more threshold limits. Such an alarm condition determination procedure based on an alarm threshold limit was described above with respect to FIG. 5 and is not repeated here.

On the other hand, if the answer to the determination at the decision state 740 is YES (e.g., the patient motion/position likely to cause significant changes, and the significant changes are not likely to have a clinical significance), the process 700 makes a detour to operation 760 in which a modification is made to the alarm condition determination procedure, e.g., by increasing the alarm threshold limits for one or more vital signs, and then proceeds to operation 770 in which the modified alarm condition determination procedure is performed by the surveillance server 60 based on the increased alarm threshold limit. For example, the alarm condition determination procedure can include determining whether the vital-sign readings have stayed above the increased alarm threshold limit for a predetermined number of samples.

In certain embodiments, the amount of increase to the alarm threshold limit is fixed (e.g., 10%). In other embodiments, the amount of increase is linearly proportional to or is another type of a function based on a number of factors including, but not limited to: 1) level of patient's physical activity as determined by the acceleration readings, 2) time duration of the physical activity; 3) category for the patient motion/position (e.g., fast walking or lying on the side in a prone position); and 4) patient specific information such as age, sex, and medical condition (present and past).

The process 700 proceeds to decision state 780 in which it is determined whether the alarm condition determination of the operation 780 indicates an existence of an alarm condition. If the answer to the determination at the decision state 780 is NO (no alarm condition exists), the process 700 loops back to the operations 710 and 720 where new sets of vital-sign readings and acceleration readings are received from the monitor patch 20B. On the other hand, if the answer to the determination at the decision state 780 is YES (an alarm condition exists), the process 700 proceeds to operation 790 in which an alarm notification is sent to the hospital system 100 and then to the operations 710 and 720 where new sets of vital-sign readings and acceleration readings are received from the monitor patch 20B.

One skilled in the art would understand in view of the present disclosure that various systems and methods described above provide a number of important benefits to the vital sign monitoring system of the present disclosure including preventing or reducing occurrences of false alarms associated with motion and/or position of the patient to whom the monitor patch is attached. In addition, the systems and methods improve the quality and reliability of vital-sign readings actually used for the alarm condition determination.

The foregoing description is provided to enable any person skilled in the art to practice the various embodiments described herein. While the foregoing embodiments have been particularly described with reference to the various figures and embodiments, it should be understood that these are for illustration purposes only and should not be taken as limiting the scope of the claims.

The word “exemplary” is used herein to mean “serving as an example or illustration.” Any aspect or design described herein as “exemplary” is not necessarily to be construed as preferred or advantageous over other aspects or designs.

A reference to an element in the singular is not intended to mean “one and only one” unless specifically stated, but rather “one or more.” The term “some” refers to one or more. Underlined and/or italicized headings and subheadings are used for convenience only, do not limit the invention, and are not referred to in connection with the interpretation of the description of the invention. All structural and functional equivalents to the elements of the various embodiments of the invention described throughout this disclosure that are known or later come to be known to those of ordinary skill in the art are expressed incorporated herein by reference and intended to be encompassed by the invention. Moreover, nothing disclosed herein is intended to be dedicated to the public regardless of whether such disclosure is explicitly recited in the above description.

What is claimed is:

1. A method of reducing false alarms or false negatives associated with vital-sign monitoring, the method comprising:
receiving one or more vital-sign readings of a patient;
receiving one or more acceleration readings from an accelerometer attached to the patient;
determining at least one of motion and position of the patient based at least in part on the one or more acceleration readings; and
modifying an alarm condition determination procedure if at least one of the motion and the position satisfies a predefined condition.

2. The method of claim 1, wherein the determining comprises determining whether the patient is in one of lying, sitting and standing positions.

3. The method of claim 1, wherein the determining comprises determining whether the patient is stationary or moving.

4. The method of claim 1, wherein, if the patient is determined to be moving, the determining further comprises determining whether the patient is ambulating or being moved by a carrier.

5. The method of claim 1, wherein the alarm condition determination procedure comprises comparing the one or more vital-sign readings to one or more threshold limits, further wherein modifying the alarm condition determination procedure comprises adjusting the one or more threshold limits.

6. The method of claim 1, wherein modifying the alarm condition determination procedure comprises excluding the one or more vital-sign readings from the alarm condition determination procedure.

7. The method of claim 1, wherein the predefined condition comprises at least one of the motion and the position indicating that the patient is engaged in a motion or is in a position that is likely to produce artifacts in or disrupt the one or more vital-sign readings, further wherein the one or more vital-sign readings are excluded from the alarm condition determination procedure if the predefined condition is satisfied.

8. The method of claim 1, wherein the predefined condition comprises the one or more acceleration readings indicating that the patient is engaged in a motion or is in a position that is likely to cause the one or more vital-sign readings to substantially change, further wherein one or more threshold limits for alarm condition determination are changed if the predefined condition is satisfied.

9. The method of claim 8, wherein an amount by which the one or more threshold limits are changed is determined based on patient-specific information, the patient-specific information comprising at least one of age, sex, and medical condition of the patient.

10. The method of claim 1, wherein the determining and the modifying are performed at a vital-sign monitor configured to monitor one or more vital signs of the patient.

11. The method of claim 1, wherein the determining and the modifying are performed at a surveillance server configured to gather data relating to one or more vital signs of the patient.

12. A vital sign monitoring system, comprising:
a vital-sign monitor configured to monitor one or more vital signs of a patient;
a surveillance server configured to gather data relating to the one or more vital signs of the patient from the vital-sign monitor;
an accelerometer attached to the patient; and
a processor in data communication with the vital-sign monitor and the accelerometer, the processor configured to:
receive one or more vital-sign readings of the patient,
receive one or more acceleration readings from the accelerometer,
determine at least one of motion and position of the patient based at least in part on the one or more acceleration readings, and
modify an alarm condition determination procedure if at least one of the motion and the position satisfies a predefined condition.

13. The system of claim 12, wherein the one or more vital-signs comprise at least one of body temperature, pulse rate, blood pressure, and respiratory rate.

14. The system of claim 12, wherein the vital-sign monitor is a vital-sign monitor patch configured to wirelessly transmit the data to the surveillance server.

15. The system of claim 14, wherein the accelerometer is part of the monitor patch.

16. The system of claim 14, where in the processor is part of the monitor patch.

17. The system of claim 14, wherein the processor is part of the surveillance server.

18. The system of claim 12, wherein the accelerometer comprises a three-axis accelerometer configured to provide three outputs responsive to accelerations in three orthogonal directions.

19. The system of claim 12, wherein the predefined condition comprises at least one of the motion and the position indicating that the patient is engaged in a motion or is in a position that is likely to cause artifacts in the one or more vital-sign readings, further wherein the one or more vital-sign readings are excluded from the alarm condition determination procedure if the predefined condition is satisfied.

20. The system of claim 12, wherein the predefined condition comprises the one or more acceleration readings indicating that the patient is engaged in a motion or is in a position that is likely to cause the one or more vital-sign readings to substantially change without having a medical significance for the patient, further wherein one or more threshold limits for alarm condition determination are changed if the predefined condition is satisfied.
